(19) United States
(12) Patent Application Publication
(10) Pub. No.: US 2021/0252117 A1
(43) Pub. Date: Aug. 19, 2021

(54) Method of Treating Cervical Vertigo

(71) Applicant: Ib Odderson, Kirkland, WA (US)

(72) Inventor: Ib Odderson, Kirkland, WA (US)

(21) Appl. No.: 17/157,787

(22) Filed: Jan. 25, 2021

Related U.S. Application Data
(60) Provisional application No. 62/964,923, filed on Jan. 23, 2020.

(51) Int. Cl. A61K 38/48; A61P 15/00

(52) U.S. Cl. CPC A61K 38/4893; A61P 15/00

(57) ABSTRACT

A method for treating cervical vertigo by injecting botulinum toxic into the upper cervical neck muscles.

This utility patent application is based on and claims the filing date benefit of U.S. Provisional Patent Application (Application No. 62/964,923) filed on Jan. 23, 2020.

Notice is given that the following patent document contains original material subject to copyright protection. The copyright owner has no objection to the facsimile or digital download reproduction of all or part of the patent document, but otherwise reserves all copyrights.

### BACKGROUND OF THE INVENTION

### Field of the Invention

This invention pertains to method for treating cervical vertigo.

### Description of the Related Art

Cervical Vertigo (CV) or Cervical Dizziness may occur after trauma to the head and neck such as whiplash injury, after overuse or have insidious onset. The underlying condition of cervical dystonia may not be readily recognized without specialized electrodiagnostic testing.

Heretofore, a patient who has had a neck injury and experiencing vertigo, but not diagnosed with cervical dystonia, is typically treated with physical therapy, vestibular therapy, acupuncture, massage, chiropractic manipulations, trigger point injections, medications, radiofrequency ablation, greater occipital nerve block, and surgery. Unfortunately, all of these treatments are associated with variable success.

What is needed is awareness, that patients who have vertigo or dizziness may have a condition such as neck muscle overactivity (CV) that can be treated effectively with botulinum toxin.

### SUMMARY OF THE INVENTION

A method of treating cervical vertigo with botulinum toxin injected into neck muscles.

### DESCRIPTION OF THE PREFERRED EMBODIMENT(S)

Disclosed herein is a method for treating cervical vertigo developed after a neck injury by injecting botulinum toxin A.

### Case Presented:

A 54 year-old woman was hit by a wave in the back approximately ten years ago. She subsequently developed neck pain, vertigo spells and muscle tension in the left trapezius. Vertigo would sometimes occur when she was lying in bed, with the first steps in the morning, and with full cervical rotation to the left. When the vertigo was severe, she felt nauseated and mostly had to lie down. She felt the dizziness was associated with tightness in the upper cervical muscles including the oblique capitis superior and the oblique capitis inferior areas.

Testing for benign paroxysmal positional vertigo (BPPV) was negative, a course of prednisone, and two cervical epidural steroid injections were given, all with short term benefits. A cervical MRI showed moderate to severe foraminal stenosis at R C3-4, and L C-6-7. Moderate foraminal stenosis at R C3-4, R C6-7, L C5-6 and L C7-T1. Mild foraminal stenosis at L C3-4.

An EMG assessment confirm dystonia in the left trapezius, levator scapula, longissimus capitis, oblique capitis inferior, and oblique capitis superior.

A diagnosis of cervical dystonia was made based on her history, physical examination, head tilt to the left, limited cervical range of motion, and cervical dystonic muscular activity on EMG examination.

She was initially injected under EMO guidance with a total 100 units of botulinum toxin A (1:2 dilution in preservative free saline) bilaterally to the oblique capitis superior muscles (OCS); (15 units+15 units) to the longissimus capitis muscle proximally; (15 units+15 units) and the trapezius (15 units) and the levator scapula distally (25 u).

She experienced consistent resolution of her vertigo symptoms and pain after each treatment for a total of five treatments. Muscles consistently injected were the OCS bilaterally.

It should be understood that although the patient was treated with five total treatments, the number of total treatment may from one treatment to an unlimited number of treatments. In most cases, the symptoms are relieved for 2 to 3 months after each treatment and additional treatments are given when vertigo returns.

It should also be pointed out that although botulinum A toxin was used, other types of botulinium toxins (e.g B-G) may be used. The optimal number of units given at each injection site may also vary from 2.5 units to 100 units.

An important finding for explaining the etiology of cervical vertigo is diagnosis of cervical dystonia which was confirmed by EMG. It is postulated that cervical muscle overactivity and associated hyperactive muscle spindles provide an altered cervical somatosensory input. The mechanism is proprioceptive, and the sensory mismatch between cervical and vestibular input would be expected to result in cervical vertigo. In the present patient, chemodenervation with botulinum toxin of the dystonic neck muscles reduced muscle spasm and pain, and thus the proprioceptive input resulting in improvement of her vertigo. Similar changes in proprioceptive input though physiotherapy, acupuncture, manipulation have been shown to reduce neck pain and dizziness. Likewise, anesthetic muscle blocks to spastic cervical muscles reduce dizziness in patents with cervical spondylosis and in patients with neck pain. Another possible explanation for improving cervical vertigo is decompression of the occipital and sub occipital nerves and blood vessels by chemodenervation of the overlying muscles. Such decompression would also promote normalization of the afferent input and possibly improve cervical vertigo.

In compliance with the statute, the invention described has been described in language more or less specific as to structural features. It should be understood however, that the invention is not limited to the specific features shown, since the means and construction shown, comprises the preferred embodiments for putting the invention into effect. The invention is therefore claimed in its forms or modifications within the legitimate and valid scope of the amended claims, appropriately interpreted under the doctrine of equivalents.

I claim:

1. A method for treating cervical vertigo in a patient comprising injecting botulinum toxin A into the patient's upper cervical muscles.

2. A method for treating cervical vertigo in a patient, comprising the following steps:
a. selecting botulinum toxin mixed in a 1:2 ratio with normal saline;
b. injecting 15 units of botulinum toxin bilaterally into the oblique capitis superior muscles;
c. injecting twice 15 units of botulinum toxin to the longissimus capitis muscle proximally;
d. injecting twice 15 units of botulinum toxin into the trapezius; and
e. injecting 25 units of botulinum toxin A into the levator scapula distally.

3. The method as recited in claim 2, wherein steps a-e are completed in a single treatment session and then repeated when cervical vertigo returns.

4. The method as recited in claim 3, wherein each subsequent treatment session includes steps a-e.

5. The method as recited in claim 4, wherein each subsequent treatment session is repeated every three months.

6. The method as recited in claim 1, wherein the botulinum toxin is botulinum toxin A.

7. A method for reducing cervical vertigo in a patient comprising, administration by injection to the cervical neck region of the patient a composition, the method comprising injecting botulinum toxin A 2.5 to 100 units to at least the following muscles: the oblique capitis superior the oblique capitis inferior muscles, the longissimus capitis muscle, the trapezius and the levator scapula.
